(19) United States
(12) Patent Application Publication
(10) Pub. No.: US 2007/0077269 A1
(43) Pub. Date: Apr. 5, 2007

(54) Method of birth control and hormone regulation

(76) Inventor: John R. Woodward, Dallas, TX (US)

Correspondence Address: Michael A. O'Neil;Michael A. O'Neil, P.C., Suite 820, 5949 Sherry Lane, Dallas, TX, 75225, US

(21) Appl. No.: 11/243,134

(22) Filed: Oct. 4, 2005

(51) Int. Cl. A61K 31/57; A61K 31/56

(52) U.S. Cl. 424/423; 514/170

(57) ABSTRACT

A method of birth control and hormone regulation comprises utilizing a vaginal ring worn continuously during each menstrual cycle. A vaginal ring delivers predetermined doses of progesterone and beta-estradiol. Beta-estradiol does not cause an increase in sex hormone binding globulin and thereby does not decrease a woman's levels of free testosterone and libido.

### TECHNICAL FIELD

This invention relates generally to birth control and hormone regulation, and more particularly to a method of birth control and hormone regulation utilizing a vaginal ring which is worn continuously during a woman's menstrual cycle.

### BACKGROUND AND SUMMARY OF THE INVENTION

Various methods of birth control and hormone regulation are practiced by women. Although hormone regulation is commonly associated with older women, women of child bearing age often require hormone regulation in addition to or in conjunction with birth control. The most common forms of medically prescribed birth control and hormone regulation comprise pills taken daily. However, more recently new birth control treatments have evolved which require less interaction including the following: patches worn by women which are changed on a weekly basis for the first three weeks of the menstrual cycle and no patch is worn during the last seven days of the menstrual cycle; and a vaginal ring worn during the first three weeks of the menstrual cycle and removed during the last seven days thereof to facilitate a menstrual period.

Previous contraception and hormone regulation treatments have been effective for contraception but have traditionally included ethinyl-estradiol in conjunction with progestogenic compounds including, but not limited to, levonorgestrel, desogestrel, norgestriel, and norelgestromin. The amount of ethinyl-estradiol has ranged from about 0.02 mg to about 0.75 mg in some cases, with higher amounts required in transdermal applications such as patches. Further, available contraception treatments do not eliminate the menstrual period at the end of each menstrual cycle and therefore cannot prevent cramps, heavy bleeding, headaches, painful bleeding, and other symptoms commonly experienced during menstrual periods.

Ethinyl-estradiol has been heretofore found to increase a woman's sex hormone binding globulin thereby causing a decrease in the woman's levels of free testosterone. Decreased levels of free testosterone result in decreased libido and sex drive.

Further, the estrogen used in contraception and hormone regulation treatments has varied. Ethinyl-estradiol has traditionally been the preferred estrogen for use in birth control treatments while estradiol, estrone, and estriol have been the preferred estrogen for hormone regulation. The difference in preferred medication results in multiple treatments—one for birth control and another for hormone regulation.

The present invention comprises a method of birth control and hormone regulation which overcomes the foregoing and other difficulties that have long since characterized the prior art. In accordance with the broader aspects of the invention, a method of birth control and hormone regulation comprises the steps of providing a vaginal ring which is worn continuously for the entire menstrual cycle. A predetermined quantity of a progestogenic compound and a predetermined quantity of beta-estradiol are provided within the vaginal ring and released therefrom.

In accordance with more specific aspects of the invention, a method of contraception and hormone replacement utilizes a vaginal ring having a predetermined quantity of a progestogenic compound and a predetermined quantity of beta-estradiol provided therein. The vaginal ring is worn continuously during a woman's entire menstrual cycle. By wearing the ring continuously throughout the entire menstrual cycle, women avoid the physical discomforts that accompany menstrual periods including cramps, headaches, heavy menstrual bleeding, and painful menstrual bleeding.

The amount of beta-estradiol administered is at least about 30 mg. Although this dosage amount is greater as compared with the amount of ethinyl-estradiol used in traditional contraceptive and hormone replacement treatments, the potency of the beta-estradiol is less than the potency of ethynl-estradiol thereby resulting in a lower dose of administered estrogen.

### BRIEF DESCRIPTION OF THE DRAWING

A more complete understanding of the present invention may be had by reference to the following Detailed Description when taken in connection with the accompanying Drawings, wherein:

FIG. 1 is a flowchart illustrating the method of the present invention.

### DETAILED DESCRIPTION

Introduction

The following example describes a method of birth control and hormone regulation.

### Example

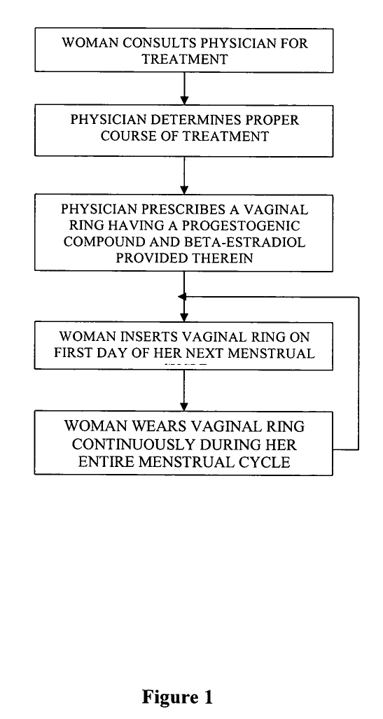

Referring to the Drawings, and particularly to FIG. 1 thereof, the steps in the method of birth control and hormone regulation of the present invention are shown therein. A woman seeking medical treatment for birth control and/or hormone regulation consults her physician. The woman may consult either her gynecologist or her primary care physician. The woman and her physician review and discuss the woman's health and any symptoms accompanying her menstrual period to determine whether the treatment method of the present invention is appropriate. If the physician determines the treatment is needed, the physician prescribes a vaginal ring having a predetermined quantity of a progestogenic compound provided therein in combination with a predetermined quantity of beta-estradiol.

On the first day of her next menstrual cycle the woman inserts the provided vaginal ring into her vagina. The woman wears the ring continuously during her entire menstrual cycle, typically about 28 days. At the end of menstrual cycle and before the next menstrual cycle begins, the woman removes the vaginal ring from her vagina and inserts a new ring therein.

The vaginal ring comprises a compartment having a permeable skin thereby enabling a controlled delivery of the drugs provided therein. Throughout the woman's menstrual cycle the ring slowly dispenses the progestogenic compound and the beta-estradiol providing contraception treatment and regulating the woman's hormones. The structure of a vaginal ring which can be used in the practice of the present invention is shown and described in U.S. Pat. No. 5,989,581, issued to Groenewegen on Nov. 23, 1999. A second vaginal ring which may be used in the practice of the present invention is shown and described in U.S. Pat. No. 6,126,958, issued to Saleh, et. al. on Oct. 3, 2000.

The predetermined quantity of progestogenic compound is within the range traditionally provided in birth control and hormone regulation, which is well known in the art, while the predetermined amount of beta-estradiol is at least about 30 mg. For example, when the progestogenic compound levonorgestrel is provided in the ring, the amount of levonorgestrel is about 0.100 mg, the same amount traditionally found in contraceptives employing levonorgestrel, and the level of beta-estradiol is about 30 mg.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of birth control practiced by a woman having a menstrual cycle comprising the steps of:
providing a vaginal ring having at least one compartment therein;
the compartment of the vaginal ring having a permeable skin enabling delivery of a drug therethrough;
providing a predetermined quantity of a progestogenic compound;
providing a predetermined quantity of beta-estradiol;
preparing a mixture comprising the predetermined quantity of the progestogenic compound and the predetermined quantity of beta-estradiol;
thereafter placing the resulting mixture within the compartment of the vaginal ring;
inserting the vaginal ring into a vagina; and
retaining the vaginal ring within the vagina of the woman continuously during her entire menstrual cycle.

2. The method according to claim 1 wherein the predetermined quantity of beta-estradiol is about 30 mg.

3. A method of hormone regulation practiced by a woman having a menstrual cycle comprising the steps of:
providing a vaginal ring having at least one compartment therein;
the compartment of the vaginal ring having a permeable skin enabling delivery of a drug therethrough;
providing a predetermined quantity of a progestogenic compound;
providing a predetermined quantity of beta-estradiol;
preparing a mixture comprising the predetermined quantity of the progestogenic compound and the predetermined quantity of beta-estradiol;
thereafter placing the resulting mixture within the compartment of the vaginal ring;
inserting the vaginal ring into a vagina; and
retaining the vaginal ring within the vagina of the woman continuously during her entire menstrual cycle.

4. The method according to claim 3 wherein the predetermined quantity of beta-estradiol is about 30 mg.

5. A method of birth control and hormone regulation practiced by a woman having a menstrual cycle comprising the steps of:
providing a vaginal ring having at least one compartment therein;
the compartment of the vaginal ring having a permeable skin enabling delivery of a drug therethrough;
providing a predetermined quantity of a progestogenic compound;
providing a predetermined quantity of beta-estradiol;
preparing a mixture comprising the predetermined quantity of the progestogenic compound and the predetermined quantity of beta-estradiol;
thereafter placing the resulting mixture within the compartment of the vaginal ring;
inserting the vaginal ring into a vagina; and
retaining the vaginal ring within the vagina of the woman continuously during her entire menstrual cycle.
